(19) United States
(12) Patent Application Publication
(10) Pub. No.: US 2021/0329853 A1
(43) Pub. Date: Oct. 28, 2021

(54) Prophylactic Mask with Mycomaterial Filter for Particulate Matter

(71) Applicant: MycoWorks, Inc., Emeryville, CA (US)

(72) Inventors: Philip Ross, San Francisco, CA (US); Matthew L. Scullin, San Francisco, CA (US); Rachel Linzer, Albany, CA (US); Ritu Bansal Mutalik, Albany, CA (US); Nicholas Romano, Berkeley, CA (US); Daniel Callahan, Berkeley, CA (US)

(21) Appl. No.: 17/242,664

(22) Filed: Apr. 28, 2021

Related U.S. Application Data
(60) Provisional application No. 63/016,899, filed on Apr. 28, 2020.

(51) Int. Cl. A01G 18/10; C12N 1/14; A41D 13/11; A01G 18/20

(52) U.S. Cl. CPC A01G 18/10; A01G 18/20; A41D 13/11; C12N 1/14

(57) ABSTRACT

A process of making a mycological biopolymer of a mycomaterial filter comprising the steps of filling a scaffold with a nutritive substrate and a fungus, placing an encasement on the scaffold to seal the scaffold, said encasement having only one outlet therein open to fresh air and defining a vacant space, incubation of the sealed scaffold at high temperatures and carbon dioxide concentrations to induce biopolymer growth into the vacant space wherein the mycological biopolymer environmental conditions comprise an environmental temperature from 55° F. to 95° F. and carbon dioxide constitutes from 2% to 8% of the environment within the vacant space, and thereafter drying the produced mycological biopolymer.

### RELATED APPLICATIONS

This application claims the benefit of U.S. provisional patent application 63/016899, filed Apr. 28, 2020, the disclosure of which is incorporated herein in its entirety.

### TECHNICAL FIELD OF THE DISCLOSURE

The present embodiment relates in general to industrial, commercial and personal protective equipment, masks, respirators, paint masks, dust masks, surgical masks or mask-like products that may be used to block particulate environmental pollutants, pathogens, and viruses, and in particular to a filter comprising mycomaterial biopolymers.

### SUMMARY OF THE INVENTION

Protective masks such as personal protective equipment masks, respirators, paint masks, dust masks, surgical masks or mask-like products are conventionally used to block environmental particulate pollutants, pathogens, and viruses. Such masks are increasingly becoming common to protect users against airborne pathogens and environmental particulates.

The present invention functions, in part, by employing various mechanical means for filtering the air passing through the mask. Said mechanical means may comprise a filter made entirely or in part out of mycomaterial. The mycomaterial can act as an antiviral coating that destroys viruses in the air passing through. The mycomaterial filter acts as an inhalation and exhalation sieve, cleansing the air as it passes through. The filter may be permanently embedded within the mask or may be included as part of a replaceable cartridge.

In some embodiments the present invention includes a breathable mask having a shape suitable for covering the user's mouth and nose and in close contact with the user's face, including a means for holding the mask in a predetermined position on the user's face and a filter including one or a plurality of layers, the filter being arranged so that a user's inhalation and/or exhalation pass through the filter, and the filter material is an aeration blended with a mycomaterial substrate. In some embodiments, the breathable mask includes a mycomaterial filter providing mechanical, electrostatic, and biological (e.g., antiviral compound-laden) filtration elements. In some embodiments, the mycomaterial filter comprises a cartridge filter or holster containing the mycomaterial filter, cartridge filter or holster being affixed to the mask securement points. In some embodiments the Ganoderma mycelium biopolymer is hydrated with glycerin or polyethylene glycol and the Ganoderma mycelium biopolymer is simultaneously configured to be in contact with a fabric.

In some embodiments the mycomaterial may be wholly or partially embedded in, coated with, or penetrated with a permanent or semi-permanent layer of antiviral or antimicrobial particles such as those including copper, silver, titanium dioxide, citric acid, or the like. In some embodiments, the Ganoderma mycelium biopolymer is hydrated with glycerin or polyethylene glycol or similar compounds. In other embodiments, the Ganoderma mycelium biopolymer is configured to be in contact with a fabric. In still other embodiments, the Ganoderma or other mycomaterial may be pure, and in other embodiments the Ganoderma or other mycomaterial may be hydrated with a suitable adjuvant, such as glycerin or polyethylene glycol, where it has been tanned (so as to crosslink the molecules within the Ganoderma and potentially change its pore size), and where any of the above are also in contact with a fabric.

In other embodiments of the invention, electrostatic means such as cloth or biosurfaces maintaining a permanent dipole are employed to filter water droplets and charged biomolecules such as viral surface proteins. Such electrostatic surfaces may extract water droplets, and the necessarily water-soluble viral particles within them, utilizing electrostatic properties. In some embodiments, the metal-retaining properties of the mycological biopolymer including the mask filter are used to form a sterilizing electrically-modulated system wherein a user may apply an electric current to the mask in order to sterilize the mask after use.

In some embodiments, a process of growing a mycological biopolymer of a mycomaterial filter is contemplated including the steps of: a) filling a scaffold with a nutritive substrate and a fungus; b) placing a encasement on the scaffold to seal the scaffold, said encasement having only one outlet therein open to fresh air and defining a vacant space; c) incubation of the sealed scaffold at high temperatures and carbon dioxide concentrations to induce biopolymer growth into the vacant space wherein the mycological biopolymer environmental conditions comprise an environmental temperature from 55° F. to 95° F. and carbon dioxide constitutes from 2% to 8% of the environment within the vacant space; and d) thereafter drying the produced mycological biopolymer. The above steps may further include the steps of placing mats in the vacant space of the scaffold and growing the mycological biopolymer about the mats in order to incorporate the mats in the mycological biopolymer and to increase tensile strength in said mycological biopolymer.

Further to the above, in some embodiments the step of incubation at high temperatures and carbon dioxide concentration occurs in the presence of a compressive pressure of at least 10 PSI to the mycological biopolymer. In other embodiments, an additional step of applying at least one morphological modifier onto the surface of the mycological biopolymer to alter the morphology of the mycelia may be included. For example, the morphological modifier may include an antiviral, hormone, gene-activating compound, calcium, and/or a calcium blocker.

In other embodiments, an additional step of compressing the mycological biopolymer to predetermined dimensions is included. This step is performed after said step of incubation and includes compressing the mycological biopolymer for an additional 0 to 72 hours to increase strength and density prior to said step of drying. In other embodiments, a step of shaping the dried mycological biopolymer to a predetermined shape is included. In other embodiments, the process of growing a mycological biopolymer for mask formation includes an additional step of sandwiching the mycological biopolymer between a pair of laminates after said step of incubation and thereafter incubating the mycological biopolymer for an additional 12 to 72 hours to adhere the mycological biopolymer between and to said pair of laminates prior to said step of drying may be included.

### BRIEF DESCRIPTION OF THE DRAWINGS

In order to enhance their clarity and improve the understanding of the various elements and embodiment shown herein, the figures have not in all cases been drawn to scale. Furthermore, elements that are known to be common and well understood to those in the industry are not depicted in order to provide a clear view of the various embodiments of the invention, thus the drawings are generalized in form in the interest of clarity and conciseness.

FIG. 1 is a side view of a mycomaterial filter for particulate matter according to a preferred embodiment of the invention.

### DETAILED DESCRIPTION OF THE EMBODIMENTS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention. Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

The present embodiment relates in general to respirators, paint masks, dust masks, or surgical masks that may be used to block particulate environmental pollutants and pathogens, and in particular to an improved filter 10 for a breathable mask, the filter 10 including in part or in total a mycomaterial filter composed of mycomaterial substrates such as a mycomaterial biopolymer. In some embodiments, the breathable mask includes a mask body 12, filter 10, and mask securement points 20. In some embodiments of the present invention, the filter provides mechanical means to dampen the velocity of airflow 16. Said mechanical means may include a mycomaterial filter 18 including, for example, a mycomaterial filter composed of a Ganoderma mycelium biopolymer formed by the process described below. In some embodiments, Ganoderma mycelium biopolymer include Ganoderma filter fibers forming honeycomb resembling structures with flexible structural components and high tensile strength. In other embodiments of the invention, electrostatic means such as cloth or biosurfaces maintaining a permanent dipole are employed to filter water droplets and charged biomolecules such as viral surface proteins. Because water itself maintains a permanent dipole, such electrostatic surfaces serve to effectively extract water droplets, and the necessarily water-soluble viral particles within them, utilizing electrostatic properties.

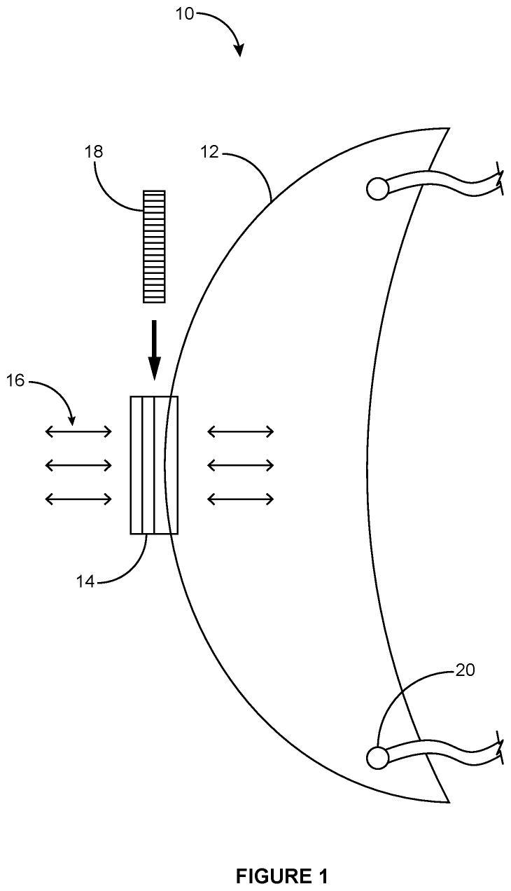

Improvements span a range of various mask (respirator) types, such as the N95 respirator, P100 respirator, various surgical masks, and/or even paper masks. Further, the present invention comprises a novel filter 10 for a mask termed a mycomaterial filter 18, which acts as anti-pathogen anti-particulate pollutant filter that has a deleterious effect on viruses in some embodiments. In one embodiment, said effect on viruses is enhanced by genetically engineering the mycelium fibers (e.g., via CRISPR and/or standard techniques known in the art) to secrete broad spectrum antibodies, antiviral compounds, and/or upregulate the natural antiviral defenses of the mycobacterial system. In some embodiments, airflow 16 including droplets transpose the mycomaterial filter 18 both during inhalation and exhalation. Notably, as shown in FIG. 1, the filter 10 may comprise a cartridge filter 14 or holster containing a mycelium filter 18, which is then affixed to mask securement points 20 proximate to the user's mouth.

Thus, before air is inhaled into the lungs of a user, the air is effectively strained by viral polar particles by the small pore size of the mycomaterial filter 18. The mycomaterial within the mycomaterial filter 10 is preferably Ganoderma lucidum (lingzhi) mycelium (e.g., a Ganoderma mycelium biopolymer), which is known to have antimicrobial and antiviral properties. In other embodiments, any Ganoderma mycelium biopolymer may be utilized. As described, the effectiveness of the mask is thus twofold—(1) as a mechanical filter due to the small pore size of the microstructure, and (2) as a reactive biological element that neutralizes microbes such as coronaviruses, potentially including the SARS-CoV-2 virus, due to the bioactivity of certain compounds in Ganoderma.

The interwoven hyphae of the present invention exhibits similar percolation and porosity properties as would be found in standard N95 masks. Through adequate thickness creating a high degree of tortuosity, the mycelial hyphae of the present invention form local pores in the size range of 1-100 μm, allowing air but disallowing particles such as a virus from fully penetrating the entire mycomaterial body. Furthermore, the mycomaterial may conform to one or more of the following standards: Bacterial Filtration Efficiency—ASTM F2101, Sub-micron Particulate Filtration Efficiency—ASTM F2299, Fluid Penetration Resistance—ASTM F1862, Breathing Resistance—MIL-M-3654C, Flammability Testing—16 CFR 1610, Biocompatibility—Irritation—ISO 10993-10, Biocompatibility—Sensitization—ISO 10993-10, Biocompatibility—Chemical Characterization—ISO 10993-18, Breathing Resistance—NIOSH 42 CFR 84.180, Particulate Filtration Efficiency—NIOSH 42 CFR 84.181.

As suggested above, in some embodiments the present invention includes a breathable mask having a shape suitable for covering the user's mouth and nose and in close contact with the user's face, including a means for holding the mask in a predetermined position on the user's face and a filter 10 including one or a plurality of layers, the filter being arranged so that a user's inhalation and/or exhalation pass through the filter. In some embodiments, the filter material is an aeration blended with a mycomaterial substrate. In some embodiments, the breathable mask includes a mycomaterial filter providing mechanical, electrostatic, and biological (e.g., antiviral compound-laden) filtration elements. In some embodiments, the mycomaterial filter comprises a cartridge filter or holster containing the mycomaterial filter, cartridge filter or holster being affixed to the mask securement points 20. Notably, as described above, in some embodiments the Ganoderma mycelium biopolymer is hydrated with glycerin or polyethylene glycol and the Ganoderma mycelium biopolymer is simultaneously configured to be in contact with a fabric.

In some embodiments the mycomaterial may be wholly or partially embedded in, coated with, or penetrated with a permanent or semi-permanent layer of antiviral or antimicrobial particles such as those including copper, silver, titanium dioxide, citric acid, or the like. In some embodiments, the Ganoderma mycelium biopolymer is hydrated with glycerin or polyethylene glycol or similar compounds. In other embodiments, the Ganoderma mycelium biopolymer is configured to be in contact with a fabric. In still other embodiments, the Ganoderma or other mycomaterial may be pure, and in other embodiments the Ganoderma or other mycomaterial may be hydrated with a suitable adjuvant, such as glycerin or polyethylene glycol, where it has been tanned (so as to crosslink the molecules within the Ganoderma and potentially change its pore size), and where any of the above are also in contact with a fabric.

In other embodiments of the invention, electrostatic means such as cloth or biosurfaces maintaining a permanent dipole are employed to filter water droplets and charged biomolecules such as viral surface proteins. Such electrostatic surfaces serve to effectively extract water droplets, and the necessarily water-soluble viral particles within them, utilizing electrostatic properties. In some embodiments, the metal-retaining properties of the mycological biopolymer including the mask filter are used to form a sterilizing electrically-modulated system wherein a user may apply an electric current to the mask in order to sterilize the mask after use.

In some embodiments, a process of growing a mycological biopolymer of a mycomaterial filter is contemplated including the steps of: a) filling a scaffold with a nutritive substrate and a fungus; b) placing a encasement on the scaffold to seal the scaffold, said encasement having only one outlet therein open to fresh air and defining a vacant space; c) incubation of the sealed scaffold at high temperatures and carbon dioxide concentrations to induce biopolymer growth into the vacant space wherein the mycological biopolymer environmental conditions comprise an environmental temperature from 55° F. to 95° F. and carbon dioxide constitutes from 2% to 8% of the environment within the vacant space; and d) thereafter drying the produced mycological biopolymer. The above steps may further include the steps of placing mats in the vacant space of the scaffold and growing the mycological biopolymer about the mats in order to incorporate the mats in the mycological biopolymer and to increase tensile strength in said mycological biopolymer.

Further to the above, in some embodiments the step of incubation at high temperatures and carbon dioxide concentration occurs in the presence of a compressive pressure of at least 10 PSI to the mycological biopolymer. The consequent compressed shape has a weight of which at most 15% of said weight is made up of water in some embodiments. In other embodiments, referencing the process of growing a mycological biopolymer for mask formation, an additional step of applying at least one morphological modifier onto the surface of the mycological biopolymer to alter the morphology of the mycelia may be included. For example, the morphological modifier may include an antiviral, hormone, gene-activating compound, calcium, and/or a calcium blocker.

In other embodiments, referencing the above process of growing a mycological biopolymer for mask formation, an additional step of compressing the mycological biopolymer to predetermined dimensions is included. This step is performed after said step of incubation and includes compressing the mycological biopolymer for an additional 0 to 72 hours to increase strength and density prior to said step of drying. In other embodiments, a step of shaping the dried mycological biopolymer to a predetermined shape is included. In other embodiments, the process of growing a mycological biopolymer for mask formation includes an additional step of sandwiching the mycological biopolymer between a pair of laminates after said step of incubation and thereafter further incubating the mycological biopolymer for an additional 12 to 72 hours to adhere the mycological biopolymer between and to said pair of laminates prior to said step of drying may be included. In some embodiments, the dried mycological biopolymer forms a predetermined shape adapted to fit a user's face in a personalized fashion.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and. arrangements. Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

Further to the above, the foregoing description of the preferred embodiment of the present invention has been presented for the purpose of illustration and description. It is not intended to be exhaustive or to limit the invention to the precise form disclosed. Many modifications and variations and permutations are possible in light of the above teachings. It is intended that the scope of the present invention to not be limited by this detailed description, but by the claims and the equivalents to the claims appended hereto.

What is claimed is:

1. A breathable mask having a shape suitable for covering the user's mouth and nose and in close contact with the user's face, comprising a means for holding the mask in a predetermined position on the user's face and a filter comprising one or a plurality of layers, the filter being arranged so that a user's inhalation and/or exhalation pass through the filter, and wherein the filter further comprises a mycomaterial substrate.

2. The mask of claim 1, wherein the breathable mask comprises a mask body, filter, and mask securement points, and wherein the filter is configured to dampen the velocity of airflow produced by a user's mouth and nose.

3. The mask of claim 2, wherein the filter further comprises a mycomaterial filter using mechanical, electrostatic, and biological filtration elements to filter the airflow of a user.

4. The mask of claim 3, wherein the mycomaterial filter comprises a cartridge filter or holster containing the mycomaterial filter, the cartridge filter or holster being affixed to mask securement points.

5. The mask of claim 2, wherein the mycomaterial filter further comprises a Ganoderma mycelium biopolymer.

6. The mask of claim 5, wherein the Ganoderma mycelium biopolymer is hydrated with glycerin or polyethylene glycol and wherein the Ganoderma mycelium biopolymer is configured to be in contact with a fabric.

7. A process of making a mycological biopolymer of a mycomaterial filter comprising the steps of:
filling a scaffold with a nutritive substrate and a fungus;
placing an encasement on the scaffold to seal the scaffold, said encasement having only one outlet therein open to fresh air and defining a vacant space;
incubation of the sealed scaffold at high temperatures and carbon dioxide concentrations to induce biopolymer growth into the vacant space wherein the mycological biopolymer environmental conditions comprise an environmental temperature from 55° F. to 95° F. and carbon dioxide constitutes from 2% to 8% of the environment within the vacant space; and
thereafter drying the produced mycological biopolymer.

8. A process as set forth in claim 7 further comprising the steps of placing mats in the vacant space of the scaffold and growing the mycological biopolymer about the mats to incorporate the mats into the mycological biopolymer and to increase tensile strength in said mycological biopolymer.

9. A process as set forth in claim 7 wherein said step of incubation at high temperatures and carbon dioxide concentration occurs in the presence of a compressive pressure of at least 10 PSI to the mycological biopolymer, and wherein the consequent compressed shape has a weight of which at most 15% of said weight is made up of water.

10. A process as set forth in claim 7 further comprising the step of applying at least one morphological modifier onto the surface of the mycological biopolymer to alter the morphology of the mycelia.

11. A process as set forth in claim 10 wherein said morphological modifier is at least one of an antiviral, hormone, gene-activating compound, calcium, and/or a calcium blocker.

12. A process as set forth in claim 7 further comprising the step of compressing the mycological biopolymer to predetermined dimensions after said step of incubation and thereafter incubating the compressed mycological biopolymer for an additional 0 to 72 hours to increase strength and density prior to said step of drying.

13. A process as set forth in claim 12 further comprising the step of shaping the dried mycological biopolymer to a predetermined shape adapted to fit a user's face in a personalized fashion.

14. A process as set forth in claim 7 further comprising the step of sandwiching the mycological biopolymer between a pair of laminates after said step of incubation and thereafter incubating the mycological biopolymer for an additional 12 to 72 hours to adhere the mycological biopolymer between and to said pair of laminates prior to said step of drying.

15. A product as made by the process of claim 7.
